(19) United States
(12) Patent Application Publication
(10) Pub. No.: US 2021/0295574 A1
(43) Pub. Date: Sep. 23, 2021

(54) MOTION COMPENSATED RECONSTRUCTION FOR HELICAL COMPUTER TOMOGRAPHY

(71) Applicant: KONINKLIJKE PHILIPS N.V., EINDHOVEN (NL)

(72) Inventors: Michael GRASS, BUCHHOLZ IN DER NORDHEIDE (DE); Axel THRAN, HAMBURG (DE); Rolf Dieter BIPPUS, HAMBURG (DE); Kevin Martin BROWN, CHARDON, OH (US)

(21) Appl. No.: 16/330,415

(22) PCT Filed: Sep. 11, 2017
(86) PCT No.: PCT/EP2017/072672

§ 371 Date: Mar. 5, 2019

(51) Int. Cl. G06T 11/00; A61B 6/00

(52) U.S. Cl. CPC G06T 11/005; G06T 11/006; G06T 2211/424; G06T 2211/421; A61B 6/527

(57) ABSTRACT

An imaging method includes obtaining projection data for a helical scan of a subject. The method further includes reconstructing, for a particular time and image slice location of interest, a first temporal motion state image at an earlier time on the detector array and offset from the central row in a first direction with projection data from a first to subset of detector rows, and reconstructing, for the particular time and image slice location, a second temporal motion state image at a later time on the detector array and offset from the central row in a second direction with projection data from a second different subset of detector rows. The method further includes estimating a distortion vector field between the first and second temporal motion state images, and constructing motion compensated volu-metric image data with a motion compensated reconstruction algorithm using the distortion vector field to compensate for arbitrary motion.

### FIELD OF THE INVENTION

The following generally relates to imaging and more particularly to compensating for motion in helical scans and is described with particular application to computed tomography (CT).

### BACKGROUND OF THE INVENTION

A CT scanner generally includes an X-ray tube mounted on a rotatable gantry that rotates around an examination region about a z-axis. The X-ray tube emits radiation that traverses the examination region and a subject or object positioned therein. An X-ray sensitive radiation detector array subtends an angular arc opposite the examination region from the X-ray tube, detects radiation that traverses the examination region, and generates a signal indicative thereof. A reconstructor processes the signal and reconstructs volumetric image data indicative of the examination region.

Subject motion during scanning leads to image artifacts such as blurring and/or other image artifacts in the reconstructed volumetric image data. Depending on the severity of the artifacts, the subject may need to be re-scanned, which increases subject dose, and ionizing radiation can cause damage to cells. Instructing the subject to hold their breath during a thorax or adnominal scan can reduce periodic motion due to the respiratory cycle. Furthermore, there are motion compensated reconstruction algorithms that compensate for some periodic motion such as that due to the cardiac cycle and/or the respiratory cycle.

Involuntary motion such as, e.g., coughing, hiccups, or bowel motion, may also occur during a helical scan and likewise can lead to blurring in the reconstructed volumetric image data. The motion pattern for involuntary motion is non-periodic. Unfortunately, the subject may not be able to prevent such motion and motion compensated reconstruction algorithms for periodic motion are not well-suited for compensating for non-periodic motion.

In addition, motion within the object can lead to distortion of the shape of the scanned object in the volumetric image data. The distortion depends on the direction of the motion. For example, motion transverse to the z-axis movement of the table supporting the object leads to shear strain in the x-z-view in the reconstructed image, motion in the direction of the table movement leads to compression in the y-z-view in the reconstructed image, and motion in the direction opposite of the table movement leads to stretching in the y-z-view in the reconstructed image.

As such, there is an unresolved need for another approach for another motion compensated reconstruction approach (e.g., one that mitigates at least the above-noted blurring and/or distortion).

### SUMMARY OF THE INVENTION

Aspects described herein address the above-referenced problems and/or others.

In one aspect, an imaging system includes an X-ray source configured to emit X-ray radiation, a two-dimensional detector array, including a plurality of rows of detectors, configured to detect X-ray radiation and generate a signal indicative thereof, and a reconstructor configured to process the signal and reconstruct volumetric imaged data corrected for arbitrary motion. The reconstructor is configured to generate at least two temporal motion state images, including a first temporal motion state image when a slice location of interest is located in a first sub-portion of the two dimensional detector array with projection data from a first subset of detector rows, and a second temporal motion state image when the slice location of interest is located in a second different sub-portion of the two dimensional detector array with projection data from a first different subset of detector rows. The reconstructor is further configured to generate a distortion vector field with the at least the first and second temporal motion state images, wherein the distortion vector field represents motion; and generate a motion compensated volumetric image data when the slice location of interest is centered on the two-dimensional detector array with the distortion vector field.

In another aspect, a computer readable medium is encoded with computer executable instructions which, when executed by a processor, causes the processor to: obtain projection data for a helical scan of a subject; reconstruct, for a particular time and image slice location of interest, a first temporal motion state image at an earlier time on the detector array and offset from the central row in a first direction with projection data from a first subset of detector rows; reconstruct, for the particular time and image slice location, a second temporal motion state image at a later time on the detector array and offset from the central row in a second direction with projection data from a second different subset of detector rows; estimate a distortion vector field between the first and second temporal motion state images, and construct motion compensated volumetric image data with a motion compensated reconstruction algorithm using the distortion vector field to compensate for arbitrary motion.

In another aspect, imaging method includes constructing three-dimensional images of different motion states from a single helical scan by applying different aperture weighting functions to an output of different subsets of detector rows of a detector of an imaging system, calculating a distortion vector field in-between the different temporal images using an image registration algorithm, and reconstructing a motion compensated image which compensates for arbitrary motion using the distortion vector field.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 diagrammatically illustrates an example imaging system with a reconstructor that employs a motion reconstruction algorithm.

FIG. 2 diagrammatically illustrates an example of the motion compensated reconstruction algorithm.

FIG. 3 depicts detector coverage of a subject for a spiral path of a focal spot during a helical scan.

FIG. 4 diagrammatically illustrates example weighing functions for generating two temporal motion state images for two different time points.

FIG. 5 diagrammatically illustrates example weighing functions for generating three temporal motion state images for three different time points.

FIG. 6 depicts a schematic view of the beam geometry in connection with determining a distortion vector field for motion.

FIG. 7 depicts a schematic view of the focal spot, voxels, and the detector array for taking a distortion vector field for object motion into account in the reconstruction.

FIG. 8 illustrates an example method for motion correction in accordance with an embodiment herein.

FIG. 9 diagrammatically illustrates example weighing functions for generating N temporal motion state images for N different time points.

FIG. 10 shows a motion corrected image with distortions from a lateral motion of the patient leading to a shear and below the shear region an elongation of the image due to object motion in the scan direction along with plotted data that indicates for which z-values significant image distortions are expected.

FIG. 11 shows the motion corrected image with a region of the motion corrected image corrected for distortion indicated by graphical indicia about the region.

FIG. 12 shows an image without distortion for comparison.

FIG. 13 illustrates an example method for both motion and distortion correction in accordance with an embodiment herein.

### DETAILED DESCRIPTION OF EMBODIMENTS

The following describes a motion compensation approach which compensates for voluntary, involuntary, periodic, non-periodic, and/or other motion. In general, with the approached described herein, the set of rows of detectors is split into two or more sub-sets (e.g., a front and a rear, a front, a center and a rear, etc.) in the z-direction. A time difference of the resulting images is induced by using these sub-sets for the reconstruction. This is in contrast to approaches in which several images, different in time, are each generated from data from all of the rows (the entire detector).

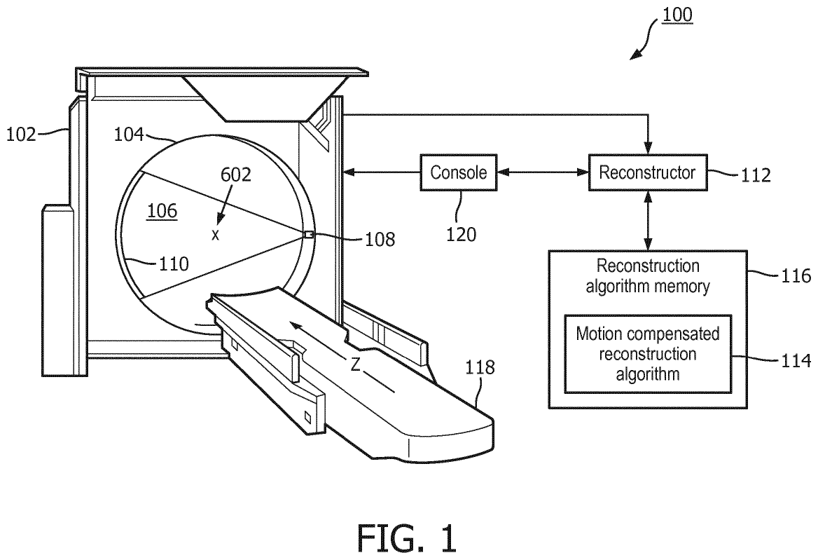

FIG. 1 diagrammatically illustrates an imaging system 100 such as a computed tomography (CT) scanner.

The system 100 includes a generally stationary gantry 102 and a rotating gantry 104. The rotating gantry 104 is rotatably supported by the stationary gantry 102 by a bearing (not visible) or the like and rotates around an examination region 106 about a z-axis, which is the axis of rotation. A radiation source 108, such as an X-ray tube, is supported by and rotates with the rotating gantry 104, and emits X-ray radiation.

A radiation sensitive detector array 110 subtends an angular arc opposite the radiation sources 108 across the examination region 106 and detects radiation traversing the examination region 106 and generates a signal (projection data) indicative thereof. The illustrated radiation sensitive detector array 110 includes a two-dimensional (2-D) array with a plurality or rows arranged with respect to each other along a direction of the z-axis.

A reconstructor 112 reconstructs the signal and generates volumetric image data indicative of the- examination region 106. The illustrated reconstructor 112 is configured to utilize, at least, a motion-compensated reconstruction algorithm 114 from the reconstruction algorithm memory 116. As described in greater detail below, the motion compensated reconstruction algorithm 114 reconstructs, for scans, two or more temporal motion state images representing different motion states for a particular image slice and then uses the two or more temporal motion state images during the reconstruction of the particular image slice. The reconstruction can mitigate motion such as voluntary motion and/or involuntary motion, e.g., due to coughing, hiccups, bowel motion, including periodic and/or non-periodic motion.

The reconstructor 112 can be implemented via hardware and/or software. For example, the reconstructor 112 can be implemented via a processor (e.g., a central processing unit or CPU, a microprocessor, a controller, etc.) configured to execute computer executable instructions stored, encoded, embedded, etc. on computer readable medium (e.g., a memory device), which excludes transitory medium, where executing the instructions causes the processor to perform one or more of the acts described herein and/or another act.

In the illustrated example, the reconstructor 112, the reconstruction algorithm memory 116 and the motion reconstruction algorithm 114 are shown as part of the imaging system 100. In another embodiment, the reconstructor 112, the reconstruction algorithm memory 116 and the motion reconstruction algorithm 114 are separate from the imaging system 100. In either instance, the reconstructor 112 and/or the reconstruction algorithm memory 116 and the motion reconstruction algorithm 114 can be local to or remote from the imaging system 100.

A support 118, such as a couch, supports a subject in the examination region 106 and can be used to position the subject with respect to x, y, and/or z axes before, during and/or after scanning A computing system serves as an operator console 120, and includes an output device such as a display configured to display the reconstructed images and an input device such as a keyboard, mouse, and/or the like. Software resident on the console 120 allows the operator to control the operation of the system 100, e.g., identifying a reconstruction algorithm, etc.

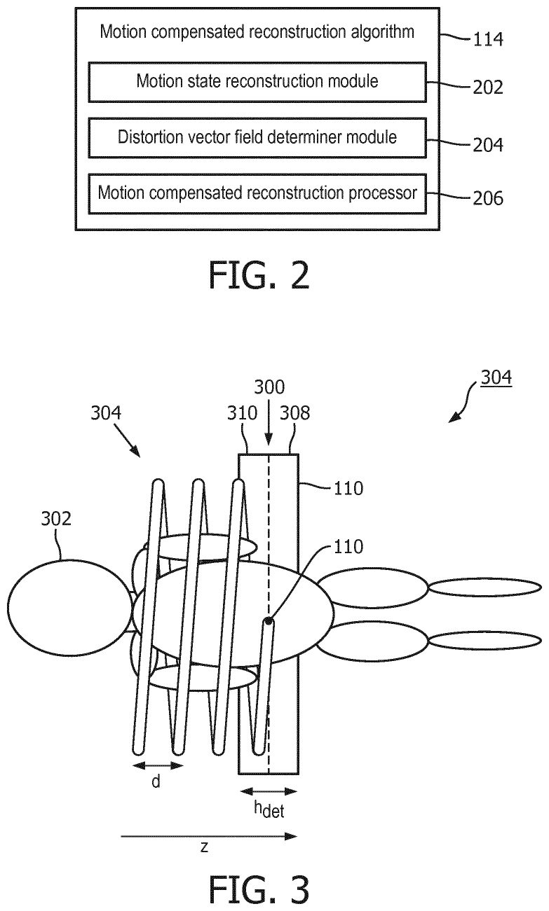

FIG. 2 diagrammatically illustrates an example of the motion compensated reconstruction algorithm 114.

In this example, the motion reconstruction algorithm 114 includes a motion state reconstruction module 202, a distortion vector field determiner module 204, and a motion compensated reconstruction processor 206. Generally, the motion state reconstruction module 202 reconstructs temporal motion state images which correspond to at least an earlier time point and a later time point relative to a current time when the source 108 is at a certain slice position. One or more other temporal motion state images for another point such as a central time point, time points between the earlier/later time points and the central time point, time points between other time points, etc. can also be reconstructed. The motion distortion field determiner module 204 computes a distortion vector field from an image registration of the temporal motion state images. The motion compensated reconstruction processor 206 employs the distortion vector field during the reconstruction of the particular slice position. The vector fields can be stored in memory along with the motion corrected slices.

The modules 202, 204 and 206 are now described in greater detail in connection with FIGS. 2-7 and 9. FIG. 3 depicts a subject 302, a spiral path 304 (with a pitch “d”) of an X-ray focus or focal spot 306 of the radiation source 108, and the detector array 110. For a particular slice position 300 having a same z coordinate as the focal spot 306 and a center of the detector array 110 at this point of time, as the rotating gantry 104 moves relative to the subject 302 in the z-direction, a first half 308 (or a first number of rows) of the detector array 110 collects data earlier in time and a second half 310 (or a second number of rows) of the detector array 110 collects data for that same slice position later in time.

In this example, the motion state reconstruction module 202 uses a segmented aperture weighting to reconstruct the temporal motion state images. The motion state reconstruction module 202 reconstructs a first temporal motion state image for the first half 308 using a first weighting function and a second image for the second half 310 using a second weighting function. The relative pitch is the relation between the pitch d and the projected detector height h_{det} 600 of FIG. 6, which is the detector height (detector extension in z-direction) projected onto the rotation axis. When only using a subset of the detector rows for the reconstruction as for the temporal motion state images, the effective detector height is reduced accordingly to the height range encompassed by the set of detector rows used for the reconstruction. So when using only half of the detector rows as in FIGS. 3-6 the relative pitch is doubled compared to the pitch calculated from the full detector height. Since images can only be reconstructed as long as the relative pitch is smaller than 2, two independent motion state images can only be reconstructed if the relative pitch d/h_{det} is equal to or less than one (1), e.g., one for the first half 308 and one for the second half 310.

Where the pitch is greater than one (1) and less than two (2), the aperture weighting function widths will be larger than half the detector height since relative pitch calculated for the fraction of the detector used for reconstruction of the motion state images is smaller than two. Thus, the weighting functions will overlap at the detector center and there will be temporal overlap, and each image will have contributions from both of the halves 308 and 310 of FIG. 6. FIGS. 2 and 6 show an example where two temporal motion state images created for halves 308 and 310. However, more than two temporal motion state images can be generated, e.g., by splitting the array 110 into more than two regions and applying suitable weighting functions.

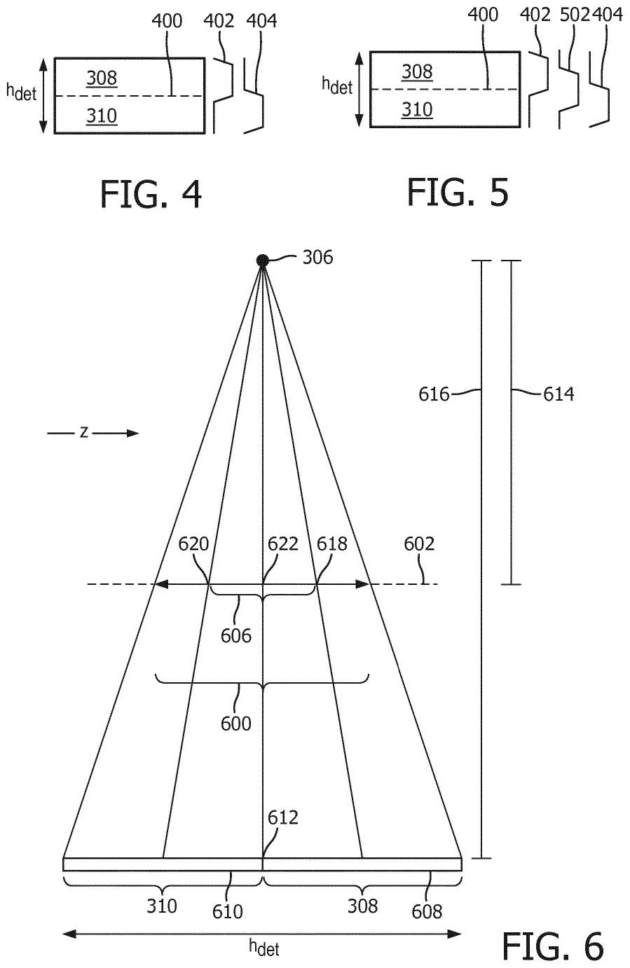

FIG. 4 shows an example of a first weighting function 402 and a second weighting function 404. A dashed line 400 represents a center of the particular slice location. The weighting function has the value 0 for the largest portion of the detector half 310 and a value of 1 for the detector half 308. Therefore, in the reconstruction of the corresponding motion state image projection data acquired with the detector half 310 will be disregarded and the image will only be reconstructed from data acquired with det. half 308. The linear transition ranges in the detector center and also at the upper detector end have the purpose to reduce image artifacts. Due to these transition ranges data from 310 near the center contribute to a small extent to the image. Vice versa the weighting function 404 is zero in 308 excluding this detector half largely from contributing to the respective motion state image.

The number and the shape of the weighting functions 402 and 404 are not limiting. An example of such weighting is discussed in Koken et al., “Aperture weighted cardiac reconstruction for cone-beam CT,” Phys. Med. Biol. 51 (2006) 3433-3448. FIG. 5 shows an example in which there are three weighting functions 402, 404 and 502 for three temporal images. The third weighting function 502 weights a central region covering a sub-portion of both halves 308 and 310 with a weight of one, and peripheral regions with a weight of zero, with an increasing weight from the peripheral region, and a decreasing weight to the peripheral region.

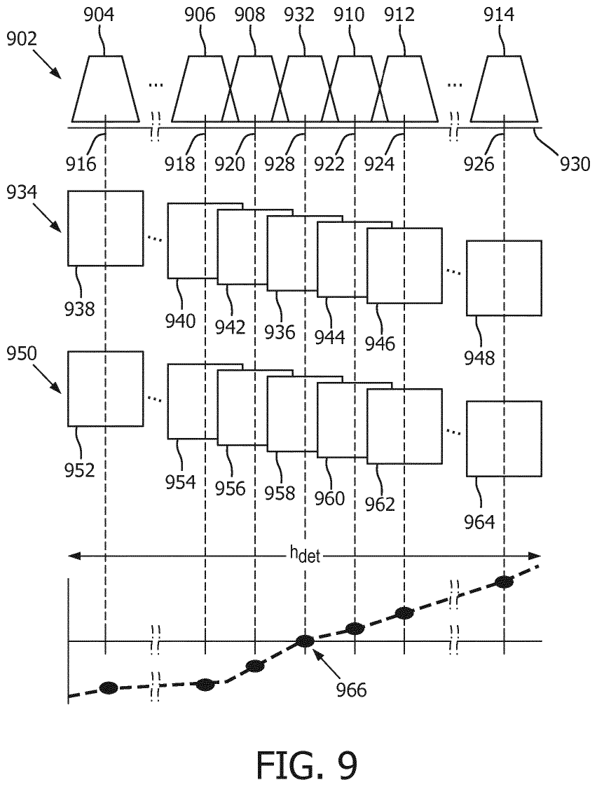

FIG. 9 shows a variation with N weighting functions 902, N−1 of which (904, 906, 908, 910, 912 and 914) are centered at non-zero detector row offsets 916, 918, 920, 922, 924 and 926 from a center 928 of a detector height range 930 and one of which (932) is centered at the center 928 of the detector height range 930. FIG. 9 also shows N corresponding motion stage images 934 reconstructed with the weighting functions, including a central 936 and N−1 other 938, 940, 942, 944, 946 and 948 motion state images. FIG. 9 also shows N distortion vector fields 950, including 952, 954, 956, 958, 960, 962 and 964, reconstructed therefrom and describing a distortion between the central 936 and the other 938, 940, 942, 944, 946 and 948 motion state images, where the central distortion vector field 958 is zero. Although FIGS. 4, 5 and 9 show trapezoidal weighting functions, non-trapezoidal weighting functions are also contemplated herein.

Returning to FIG. 2, the distortion vector field determiner module 204 calculates a distortion vector field in-between the different temporal images. This can be achieved with an image based elastic registration method, a rigid registration approach or a model based segmentation with subsequent distortion vector field interpolation. For the two images generated in connection with FIG. 4, the distortion vector field Δ\vec{r₀} is between the two temporal motion state images. For the three images generated in connection with FIG. 5, a first distortion vector field is generated between the image for the first half 308 and the central image, and a second distortion vector field is generated between the image the second half 310 and the central image. Likewise, from the distortion vector field generated based on only two aperture weighting functions depicted in FIG. 4, an equivalent set of two distortion vector fields can be generated assuming a constant motion of the object over time by multiplying the distortion vector field by +½ and by −½, respectively.

How the different aperture weighting functions relate to time differences in the motion state images is described next. FIG. 6 shows the focal spot 306, an axis of rotation 602, the first and second detector halves 308 and 310, rays of a beam impinging center regions of the first and second detector halves 308 and 310, and an average difference (Δz) 606 in the z direction of the two rays. This difference represents the average difference in z of x-ray paths corresponding to line integral values acquired with the first and second detector halves 308 and 310. Due to the constant table speed (or subject support speed) v_{T} this average difference in z corresponds to a time difference of ΔT=Δz/v_{T}.

The distortion vector field is used to correct for motion artifacts in the motion compensated reconstruction. A comparable example is discussed in Stevendaal et al., “A motion-compensated scheme for helical cone-beam reconstruction in cardiac CT angiography,” Med. Phys. 35, 3239 (2008). This reference describes, how to take a given distortion vector for an image voxel into account in the reconstruction in order to compensate for impacts of the object motion on the reconstructed image. However, in Stevendaal a periodic/cyclic motion of the subject is assumed. Its aim is to generate an image representing the object at a certain point of time or more precisely at a certain heart phase. In contrast, the image generated with the method described herein yields a three-dimensional (3-D) image, where each image slice at a certain z-coordinate represents the object at the time the x-ray focal spot had the same z-position.

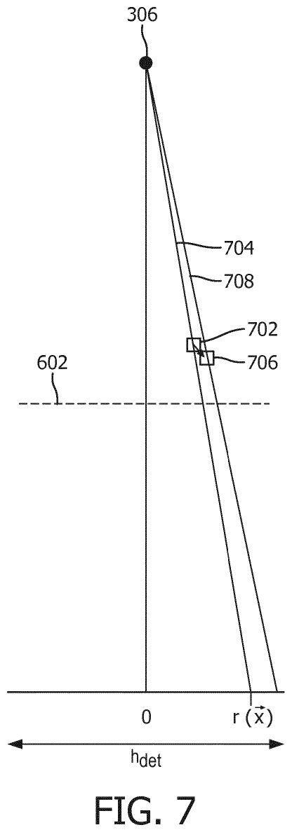

Returning to FIG. 2, the motion compensated reconstruction processor 206 employs a reconstruction algorithm (e.g., filtered back projection or iterative) which compensates for the estimated motion during the back projection process. The distortion vector field used during the back projection is zero for line integral values which are back projected from a central detector row of the detector array 110, linearly increases from zero with increasing row distance r(\vec{x}) in FIG. 7 from the central row to the row positions located at the center of the aperture weighting functions, is the estimated distortion vector field at the row positions located at the center of the aperture weighting functions, is interpolated for rows between row positions located at the centers of the aperture weighting functions and extrapolated for rows further away from the center than the outermost centers of the aperture weighting functions. This is shown in FIG. 9 at 966 for one element of the distortion vector fields.

FIG. 7 depicts an approach to compensate for object motion in the reconstruction. While reconstructing a value of voxel 702 the row distance from the detector center r(\vec{x}) is determined for each line integral value to be backprojected. Based on this value the corresponding distortion vector is determined by inter- or extrapolation of distortion vector fields as described above, and then detector data are taken into account corresponding to a voxel position 706 shifted by the determined distortion vector field, i.e., the detected x-ray intensity corresponding to a line integral along the x-ray path 708 is evaluated rather than the x-ray intensity related to line integral along the x-ray path 704 of the original voxel position.

Instead of identifying the motion state images and the corresponding distortion vector fields with row positions of centers of the aperture functions 608 and 610 (FIG. 6) and using the row positions of x-ray paths through the voxel being reconstructed as the relevant measure to sample the distortion vector fields (r(x) in FIG. 7), it is also possible to identify the motion state images with the projected detector offsets of the corresponding aperture functions and use for each line integral value to be backprojected the distance in z between the focal spot (having a z-coordinate z_{FS} for the x-ray path corresponding to the line integral value) and the voxel (with z-coordinate zᵥₒₓ) being reconstructed, i.e., zᵥₒₓ−z_{FS} as the relevant measure. The projected detector offsets are the offsets measured from the detector center projected onto the gantry axis of rotation, i.e., the differences of positions 608 and 622, scaled by a factor

$$\frac{source axis distance 614}{source detector distance 616}$$

### (FIG. 6).

An example motion compensated reconstruction is described in patent U.S. Pat. No. 8,184,883 B2, filed Nov. 14, 2006, and entitled “Motion compensated CT reconstruction of high contrast objects,” the entirety of which is incorporated herein by reference. The approach described herein mitigates motion due to voluntary and/or involuntary periodic and/or non-periodic motion, including motion due to coughing, hiccups, or bowel motion. It can be used to recover scans in which the subject coughed, breathed, and/or had other motion, and thus avoid a need to re-scan and subject the patient to additional dose. This may be valuable particularly for young children or lung screening patients. The motion compensated reconstruction algorithm described herein can be used with other motion compensated reconstruction algorithms.

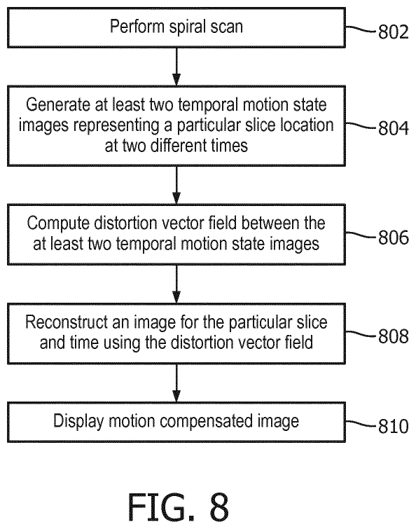

FIG. 8 illustrates an example method in accordance with an embodiment herein.

The ordering of the following acts is for explanatory purposes and is not limiting. As such, one or more of the acts can be performed in a different order, including, but not limited to, concurrently. Furthermore, one or more of the acts may be omitted and/or one or more other acts may be added.

At 802, a spiral scan is performed.

At 804, at least two temporal motion state images are generated for a particular slice location and time at two different times, as described herein.

At 806, a distortion vector field is determined from the at least two temporal motion state images, including a first temporal motion state image at an earlier point in time relative to the time, and a second temporal motion state image at a later point in time relative to the time, as described herein.

At 808, an image is generated with the acquired data using the distortion vector field to mitigate motion artifact, as described herein.

At 810, the motion compensated image is displayed.

The above may be implemented by way of computer readable instructions, encoded or embedded on computer readable storage medium, which, when executed by a computer processor(s), cause the processor(s) to carry out the described acts. Additionally or alternatively, at least one of the computer readable instructions is carried by a signal, carrier wave or other transitory medium.

The volumetric image data can also be corrected for the distortion due to the direction of motion within the object relative to movement of the subject support 118 (FIG. 1) in a helical scan based on the vector fields described above. The following describes an example non-limiting approach for doing so.

In FIG. 9, the vector fields 950 can be represented through \vec{m}ⱼ, j=−n, . . . , n, where \vec{m}ⱼ represents motion within the object during time intervals jΔt_{D} for 2n+1 vector fields, where Δt_{D} represents a mean time difference between acquisitions of two short scan images (e.g., 940 and 942 in FIG. 9) reconstructed from neighboring aperture functions (e.g., 906 and 908 being centered at different detector row offsets 918 and 920 in FIG. 9, respectively). The mean time difference can be expressed as shown in EQUATION 1:

$$Δ {t}_{D} = Δ {h}_{het} \cdot \frac{{r}_{FA}}{{r}_{FD} \cdot {v}_{t}} , \qquad EQUATION 1$$

where Δhₕₑₜ represents a distance between aperture weighing functions measured on a physical detector of the array 110, r_{FA} represents a distance between an x-ray focal spot of the source 108 and a rotation axis 602 of the imaging system 100, r_{FD} represents a distance between the x-ray focal spot and the detector, and vₜ represents a subject support speed. r_{FA} and r_{FD} are are known and constant for an imaging system, and vₜ can be obtained from the table speed scan parameter in the plan of a scan.

That is, for a transversal slice with z-coordinate z, the distortion vector field \vec{m}ⱼ(x, y, z) represents the motion in the object from the point of time t (z) where the x-ray focal spot had the same z-location and the time t(z)−jΔt_{D}, which is the mean acquisition time of short scan image number j. Since the short scan image number “0” is acquired with aperture function centered on the detector and the therefore also centered at the focal spot z-position, it is used as reference image for the distortion vector field estimation and the distortion vector fields related to it vanishes. In order to transform the motion corrected image I(x,y,z) into an undistorted image representing the object around a certain z-coordinate z₀ at the time point t₀, the reconstructor 112, in one non-limiting instance, executes the below described algorithm.

In one non-limiting example, z₀ is the z-coordinate of an image slice and represents the object at the time t₀, and z′ is the z-coordinate of a neighboring slice in the motion corrected image and represents the object at the time Δt′, where Δt′=(z′−z₀)/vₜ. To correct the motion within the time interval Δt′, i.e., to undo the motion within this time interval, the reconstructor 112 determines the corresponding distortion vector field related to −Δt′ by interpolating the distortion vector fields used for the motion compensated reconstruction. For example, the reconstructor 112 can employ a linear interpolation by finding j so that jΔt_{D}≤−Δt′<(j+1)Δt_{D} and then calculating the interpolated vector field as shown in EQUATION 2:

$${\vec{m}}_{{z}_{0}} ( x , y , {z}^{′} ) = \frac{( j + 1 ) Δ {t}_{D} - Δ {t}^{′}}{Δ {t}_{D}} {\vec{m}}_{j} ( x , y , {z}^{′} ) + \frac{Δ {t}^{′} - j Δ {t}_{D}}{Δ {t}_{D}} {\vec{m}}_{j + 1} ( x , y , {z}^{′} ) . \qquad EQUATION 2$$

Other linear and/or a non-linear interpolation can alternatively be used.

Similarly, the reconstructor 112, for time differences |Δt′|>n·Δt_{D}, performs an extrapolation. As a result, a three-dimensional distortion vector field \vec{m}ⱼ(x,y,z′) is constructed for all image slices with z-coordinates z′

$$∈ [ {z}_{0} - \frac{{h}_{D}}{2} , {z}_{0} + \frac{{h}_{D}}{2} ] .$$

This vector field is referred to herein as a “distortion correction vector field.” In one instance, this approach is limited to this z-range around z₀ since only here the distortion vector field is estimated with interpolation and/or extrapolation. The parameter z₀ can be chosen freely, thus the reconstructor 112 can calculate an undistorted image for the neighborhood of any chosen location. The undistorted image is then generated by warping the motion compensated image I(x,y,z) with the distortion correction vector field \vec{m}_{zo}:I_{undistored}(\vec{x})=I(\vec{x}+\vec{m}_{zo}(\vec{x})), where \vec{x}=(x,y,z).

The distortion, and thus also the undistorted image, will change with z₀, so the limits of the correction in +z and −z-direction, i.e.,

$${z}_{0} \pm \frac{{h}_{D}}{2}$$

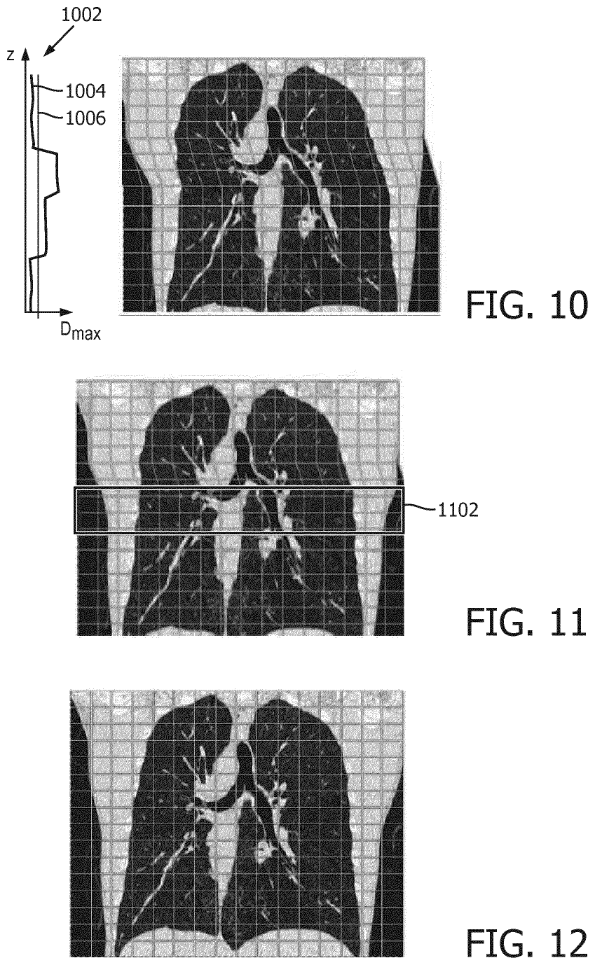

should be indicated in the image viewer. When starting the image viewer in coronal, sagittal, or 3-D mode, the motion corrected image is initially displayed. FIG. 10 shows an example of a motion corrected image with distortion. Then, a user specifies, marks and/or otherwise indicates (e.g., via a mouse click, etc.) a z₀ for which the undistorted image is calculated and displayed. The distortion correction is then applied to the region, and the distortion corrected motion compensated image is displayed. FIG. 11 shows a non-limiting example in which the distortion corrected region of the motion compensated image is indicated with a box 1102. Other indicia can be used, and/or the image can be displayed without indicating the region.

The amount of the distortion due to object motion for the region can be calculated from the distortion vector fields, as described herein. In this case it is the distortion vector fields which should be used, since they are available without additional processing.] This may be used to indicate in which image regions distortions are present and where the described correction method should be applied. An example is shown in FIG. 10 at 1002, which shows a plot 1004 of a magnitude of the distortion along z with a threshold 1006 used to indicate whether correction should be applied (e.g., only when the magnitude exceeds the threshold). An example metric indicating a significant distortion in a slice z is a maximum of the norm of all vectors in one of the distortion vector fields \vec{m}ⱼ, j≠0 as shown in EQUATION 3:

$${D}_{max}^{j} ( z ) = \frac{{max}_{x , y} ‖ {\vec{m}}_{j} ( x , y , z ) ‖ ( j + 1 ) Δ {t}_{D} - Δ {t}^{′}}{\vert j \vert Δ {t}_{D}} . \qquad EQUATION 3$$

where the maximum norm of the distortion correction vector field is scaled with the corresponding absolute time difference |j|Δt_{D} in order to convert it to a measure for the maximum speed within the object. Instead of using only one distortion vector field \vec{m}ⱼ for the metric one may also use several, for example by averaging Dₘₐₓ^{j} for all j≠0.

The console 120 can compute this metric and recommend the distortion correction if the metric (speed) exceeds a predetermined threshold. An example threshold is 10% of the table speed. Additionally or alternatively, the user manually or the console 120 can automatically activate the distortion correction whenever a geometrical measurement at a specific image location, such as volume of a nodule or extent of a lesion, is requested.

In one embodiment, both values of a measurement, corrected and uncorrected, are displayed. The console 120 can also compare these values and attach a reliability value to the measurement, e.g. indicating an unreliable measurement in case of large discrepancy and/or large local motion. In another embodiment, and as shown in FIG. 11, the undistorted image plus the motion corrected image outside the z-range of the undistorted image can additionally or alternatively be displayed. To achieve a smooth transition between both the motion corrected image parts in regions

$$z < {z}_{0} - \frac{{h}_{D}}{2} and z > {z}_{0} + \frac{{h}_{D}}{2}$$

are transformed with a same distortion correction vector field as the outermost slices of the undistorted image, i.e., by using the distortion correction vector fields

$${\vec{m}}_{{z}_{0}} ( x , y , {z}_{0} - \frac{{h}_{D}}{2} ) and {\vec{m}}_{{z}_{0}} ( x , y , {z}_{0} + \frac{{h}_{D}}{2} ) .$$

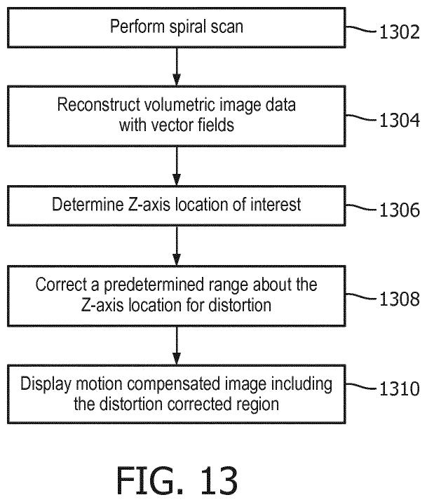

FIG. 13 illustrates an example method in accordance with an embodiment herein.

The ordering of the following acts is for explanatory purposes and is not limiting. As such, one or more of the acts can be performed in a different order, including, but not limited to, concurrently. Furthermore, one or more of the acts may be omitted and/or one or more other acts may be added.

At 1302, a spiral scan is performed.

At 1304, volumetric image data is generated using a vector field to mitigate motion artifact, as described herein and/or otherwise.

At 1306, a z-axis location of interest is determined for the volumetric image data, as described herein and/or otherwise.

At 1308, a predetermined region about the z-axis location is corrected for distortion with a distortion correction vector field, as described herein.

At 1310, a motion corrected image with the distortion corrected region is displayed.

The above may be implemented by way of computer readable instructions, encoded or embedded on computer readable storage medium, which, when executed by a computer processor(s), cause the processor(s) to carry out the described acts. Additionally or alternatively, at least one of the computer readable instructions is carried by a signal, carrier wave or other transitory medium.

In helical CT, other weighting schemes may be used and adapted to achieve the same effect as the aperture weighting as described here for the reconstruction of the motion state images. An example is illustrated in Grass, et al., “Helical cardiac cone beam reconstruction using retrospective ECG gating,” Phys. Med. Biol. 48 (2003) 3069-3084. In Grass, the illumination window is introduced, which describes for each voxel the time period it is exposed to x-rays in the helical scan. Let Φ_{f} and Φₗ be the first and the last projection in which a voxel is illuminated. For sake of convenience it is assumed here that the voxel located on the gantry axis of rotation. Then, for example, using only projections from the first half of the illumination window, i.e., from projections Φ_{f} to ½(Φ_{f}+Φₗ) by designing the projection based weighting function accordingly yields the same voxel value in the reconstructed image as the aperture weighting function 402 taking into account only line integral values measured with the front half of the detector. In general, by using a dedicated voxel dependent weighting scheme the same motion state images can be achieved as with the detector aperture weighting described before. Therefore, detector aperture weighting here refers to weighting schemes which can be translated to or have the same impact as the aperture weighting functions described here.

The approach described herein may also be applied iteratively, i.e., the distortion vector fields computed as described above may be used for motion compensated reconstruction of a second set of motion state images. When the motion compensation works perfectly, these second set of motion state images will not show any differences, since all object motions are cancelled out. However, an incomplete motion compensation will lead to differences in the second set of motion state images. The second set distortion vector fields determined from this second set of motion state images describes the remaining object motion not compensated in the first iteration. Therefore, the sums of the first distortion vector fields and the second distortion vector fields describe the object motion better and can be used for an improved motion-compensated reconstruction. They can also be used for the reconstruction of a third set of motion state images serving as input for a third iteration.

The approach described herein can be applied with spectral CT (e.g., a photon counting detector, multi-layer detector, etc. and/or phase contrast CT. For these, preprocessed projection data (e.g., projection data quantifying iodine or other contrast agents) may be used for the estimation of the distortion vector fields and the latter may be used for all image types in the motion compensated reconstruction.

With the approach describe herein, the type of object motion corrected is arbitrary (periodic, non-periodic, etc.). The motion state images are generated from different subsets of detector rows. The motion vector field dependence in reconstruction depends on the detector row hit by the x-ray path corresponding to the line integral through the voxel being reconstructed or difference of z-coordinate of voxel being reconstructed and the z-coordinate of the focal spot (i.e., the origin of the x-ray path). The resulting image displays each object slice at its state when the focal spot had the same z-coordinate. This is in contrast to a conventional approach (e.g., Stevendaal) in which the type of object motion corrected is cyclic, the motion state images are generated from different sets of whole projections acquired at different times/heart phases, the motion vector field dependence in reconstruction depends on time/heart phase, and the resulting image displays an object at one point of time/a certain heart phase.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. An imaging system, comprising:
an X-ray source configured to emit X-ray radiation;
a two-dimensional detector array, including a plurality of rows of detectors, configured to detect X-ray radiation and generate a signal indicative thereof; and
a reconstructor configured to process the signal and reconstruct volumetric imaged data corrected for arbitrary motion,
wherein the reconstructor is configured to generate a first set of at least two temporal motion state images, including a first temporal motion state image when a slice location of interest is located in a first sub-portion of the two dimensional detector array with projection data from a first subset of detector rows, and a second temporal motion state image when the slice location of interest is located in a second different sub-portion of the two dimensional detector array with projection data from a first different subset of detector rows; and
wherein the reconstructor is configured to generate a distortion vector field with the at least the first and second temporal motion state images, wherein the distortion vector field represents motion; and generate motion compensated volumetric image data when the slice location of interest is centered on the two-dimensional detector array with the distortion vector field.

2. The imaging system of claim 1, wherein the reconstructor is configured to generate at least one subsequent set of at least two temporal motion state images, at least one subsequent distortion vector field for the at least one subsequent set, and at least one set of subsequent motion compensated volumetric image data with the at least one subsequent distortion vector field.

3. The imaging system of claim 1, wherein the reconstructor generates the distortion vector field based on a distortion field determined by registering the first and second temporal motion state images.

4. The imaging system of claim 1, wherein the reconstructor generates the distortion vector field for a line integral value based on a difference between a location of a voxel in a z-direction and a location of a focal spot of the X-ray source in the z-direction corresponding to the line integral value.

5. The imaging system of claim 1, wherein the reconstructor generates the distortion vector field for a line integral value based on a distance between a middle detector row and the detector row the x-ray path corresponding to the line integral value hits the detector.

6. The imaging system of claim 1, wherein the reconstructor employs a first aperture weighting function to reconstruct the first temporal motion state image and a second aperture weighting function to reconstruct the second temporal motion state image.

7. The imaging system of claim 6, wherein the reconstructor reconstructs one or more other temporal motion state images respectively for one or more other sub-sets of the plurality of rows of detectors respectively using one or more other aperture weighting functions and estimates motion vector fields between the temporal motion state images.

8. The imaging system of claim 7, wherein the reconstructor applies the distortion vector fields respectively to row positions located at a center of a respective aperture weighting functions.

9. The imaging system of claim 7, wherein the reconstructor linearly increases a distortion vector field from a value of zero to the respective distortion vector field for line integral values back projected away from a central detector row to the row positions at the center of the respective aperture weighting function as a function of a distance from the central detector row.

10. The imaging system of claim 9, wherein the reconstructor extrapolates distortion vector fields for rows beyond the respective aperture weighting functions.

11. The imaging system of claim 9, wherein the reconstructor applies a distortion vector field of zero for line integral values back projected from the central detector row of the detector array.

12. The imaging system of claim 1, wherein the reconstructor is further configured to correct distortion of the image of a scanned object in a sub-portion of the motion compensated volumetric image data, which is due to an object motion utilizing the distortion vector fields.

13. The imaging system of claim 11, wherein the sub-portion is a predetermined range about a z-location of interest.

14. The imaging system of claim 13, wherein the reconstructor determines the predetermined range by computing a product of a height of a detector row in a z-axis direction and a ratio of a distance between an X-ray focal spot of the X-ray source and a rotation axis of the detector array to a distance between the X-ray focal spot and a detector.

15. The imaging system of claim 12, wherein the reconstructor corrects the sub-portion for the distortion by warping the sub-portion of the motion compensated image.

16. The imaging system of claim 15, wherein the reconstructor computes a distortion correction vector field by interpolating and scaling the distortion vector fields and warps the sub-portion with the corresponding vector field to undo motion within the time interval.

17. A computer readable medium encoded with computer executable instructions which when executed by a processor causes the processor to:
obtain projection data fora helical scan of a subject;
reconstruct, for a particular time and image slice location of interest, a first temporal motion state image at an earlier time on a detector array and offset from a central row in a first direction with projection data from a first subset of detector rows;
reconstruct, for the particular time and image slice location, a second temporal motion state image at a later time on the detector array and offset from the central row in a second direction with projection data from a second different subset of detector rows; and
estimate a distortion vector field between the first and second temporal motion state images; and
construct motion compensated volumetric image data with a motion compensated reconstruction algorithm using the distortion vector field to compensate for arbitrary motion.

18. The computer readable medium of claim 17, wherein the detector array includes a plurality of rows of detectors, and the computer executable instructions further cause the processor to:
reconstruct the first temporal motion state image for a first sub-set of the plurality of rows of detectors; and
reconstruct the second temporal motion state image for a second different sub-set of the plurality of rows of detectors.

19. The computer readable medium of claim 18, where the computer executable instructions further cause the processor to:
employ a first aperture weighting function to reconstruct the first temporal motion state image; and
employ a second aperture weighting function to reconstruct the second temporal motion state image.

20. The computer readable medium of claim 19, where the computer executable instructions further cause the processor to:
apply the estimated distortion vector field to row positions located at centers of the aperture weighting functions.

21. The computer readable medium of claim 20, where the computer executable instructions further cause the processor to:
linearly increase the estimated distortion vector field from a value of zero to the estimate for line integral values back projected away from a central detector row to the row positions at the centers of the aperture weighting functions as a function of a distance from the central detector row, wherein the voxel is reconstructed from all detector rows, and the distortion vector field applied depends on the projection that is backprojected.

22. The computer readable medium of claim 20, where the computer executable instructions further cause the processor to:
apply a distortion vector field of zero for line integral values back projected from the central detector row of the detector array; and
extrapolate the distortion vector field for rows outside of the row positions located at the center of the aperture weighting functions.

23. The computer readable medium of claim 15, where the computer executable instructions further cause the processor to: correct a sub-portion of the motion compensated volumetric image data for distortion of a shape of a scanned object in the motion compensated volumetric image data based on a distortion correction vector field for a same time interval.

24. The computer readable medium of claim 23, where the computer executable instructions further cause the processor to: display only the undistorted sub-portion.

25. The computer readable medium of claim 23, where the computer executable instructions further cause the processor to: display the undistorted sub-portion along with distorted portions.

26. The computer readable medium of claim 25, where the computer executable instructions further cause the processor to: visually highlight the undistorted sub-portion.

27. A computer-implemented method, comprising:
constructing three-dimensional images of different motion states from a single helical scan by applying different aperture weighting functions to an output of different subsets of detector rows of a detector of an imaging system;
calculating a distortion vector field in between the different temporal images using an image registration algorithm; and
reconstructing a motion compensated image which compensates for arbitrary motion using the distortion vector field.

28. The computer-implemented method of claim 27, further comprising:
performing the single helical scan with a pitch equal to or less than one.

29. The computer-implemented method of claim 27, further comprising:
employing one of an elastic registration, a rigid registration, or a model based segmentation with subsequent distortion vector field estimation to generate the distortion vector field.

30. The computer-implemented method of claim 27, further comprising:
receiving a signal indicating a sub-portion of the motion compensated image is to be undistorted using the distortion vector field; and
correcting the sub-portion based on the distortion vector field.

31. The computer-implemented method of claim 30, where the signal is a user input.

32. The computer-implemented method of claim 30, further comprising:
automatically generating a distortion metric to indicate where in the image the distortion is significant and should be corrected.

33. The computer-implemented method of claim 32, further comprising:
computing the distortion metric as a scaled maximum of a norm of all distortion correction vectors in the distortion correction vector fields.

34. The computer-implemented method of claim 30, further comprising:
measuring a geometry of tissue of interest in the undistorted sub-portion;
measuring a geometry of the tissue of interest in the distorted sub-portion;
determining a difference in the geometry; and
computing a reliability metric based on the difference, wherein a difference less than a predetermined threshold indicates a reliable measurement and a difference greater than the predetermined threshold indicates an unreliable measurement.
